(19) United States
(12) Patent Application Publication
(10) Pub. No.: US 2010/0114119 A1
(43) Pub. Date: May 6, 2010

(54) Tacking Device

(76) Inventors: Tyler Evans McLawhorn, Winston-Salem, NC (US); Scott T. Moore, S. King, NC (US)

Correspondence Address: BRINKS HOFER GILSON & LIONE/CHICAGO/COOK, PO BOX 10395, CHICAGO, IL, 60610, US

(21) Appl. No.: 12/608,621

(22) Filed: Oct. 29, 2009

Related U.S. Application Data
(60) Provisional application No. 61/111,074, filed on Nov. 4, 2008.

(51) Int. Cl. A61B 17/10; A61B 17/08

(52) U.S. Cl. 606/139; 606/151

(57) ABSTRACT

The present embodiments provide apparatus and systems suitable for coupling a graft member to tissue. A wire having a proximal end and a distal end are provided, each having a delivery state suitable for delivery and further comprising a deployed state. In the deployed state, the distal end is configured to engage tissue at a first location, and the proximal end is configured to engage the graft member to secure the graft member to the tissue. Optionally, a loop member may be provided for receiving a suture for further securing the graft member to the tissue. The present embodiments may also be used without a graft member to close openings in tissue.

### CROSS-REFERENCE TO RELATED APPLICATIONS

This application claims the benefit of U.S. Provisional Patent Application No. 61/111,074 filed on Nov. 4, 2008, entitled “TACKING DEVICE,” the entire contents of which are incorporated herein by reference.

### BACKGROUND

The present embodiments relate generally to medical devices, and more particularly, to apparatus and systems for coupling a graft member to tissue.

Perforations in tissue or bodily walls may be formed intentionally or unintentionally. For example, an unintentional ventral abdominal hernia may be formed in the abdominal wall due to heavy lifting, coughing, strain imposed during a bowel movement or urination, fluid in the abdominal cavity, or other reasons.

Intentional perforations may be formed, for example, during surgical procedures such as translumenal procedures. In a translumenal procedure, one or more instruments, such as an endoscope, may be inserted through a visceral wall, such as the stomach wall. During a translumenal procedure, a closure instrument may be used to close the perforation in the visceral wall. Depending on the structure comprising the perforation, it may be difficult to adequately close the perforation and prevent leakage of bodily fluids.

Attempts to seal perforations have been attempted by coupling a graft member to tissue. For example, during hernia repair, a graft material such as a mesh or patch may be disposed to cover the perforation. The graft material may completely overlap with the perforation, and the edges of the graft material may at least partially overlap with tissue surrounding the perforation. The graft material then may be secured to the surrounding tissue in an attempt to effectively cover and seal the perforation.

In order to secure the graft material to the surrounding tissue, sutures commonly are manually threaded through the full thickness of the surrounding tissue. In the case of a ventral abdominal hernia, the sutures may be threaded through the thickness of the abdominal wall, then tied down and knotted. However, such manual suturing techniques may be time consuming and/or difficult to perform.

In addition to covering and sealing perforations, there are various other instances in which it may be desirable to couple a graft material to tissue. For example, it may become desirable to couple the graft material to a region of tissue for purposes of reconstructing the local tissue. An exemplary tacking device used to couple graft material to tissue is described in U.S. Application No. 61/047,293 filed Apr. 23, 2008, the disclosure of which is incorporated herein by reference in its entirety. Likewise, there are other instances where tacking devices may be used without a graft, such as for directly closing an opening in tissue. Exemplary methods for closing an opening in tissue are described in U.S. application Ser. No. 12/557,232 (Attorney Docket No. 10000-1692) filed Sep. 10, 2009, and U.S. application Ser. No. 12/557,204 (Attorney Docket No. 10000-1681) filed Sep. 10, 2009, the disclosures of which are incorporated herein by reference in their entirety.

### SUMMARY

The present embodiments provide apparatus and systems suitable for coupling a graft member to tissue or closing an opening in tissue. In one embodiment, a tacking device is provided comprising a wire having a proximal end and a distal end. The proximal and distal ends each have delivery states suitable for delivery to a target site, and further each comprise deployed states. The distal end is configured to engage tissue at a first location in the deployed state, and the proximal end is configured to engage the graft member in the deployed state to secure the graft member to the tissue.

In one embodiment, the wire may comprise an S-shape in the deployed state. The S-shape includes configurations where the proximal and distal ends of the wire are preferably curved, and the ends of the wire extend laterally away from each other in different directions in the deployed state. The degree of curvature of each end may range anywhere from about 90 degrees to about 360 degrees. When the curvature of the wire approaches 180 degrees, an elongated “S” is formed, and the wire forms a more compact “S”, or figure-eight shape, when the curvature is about 360 degrees. The wire may comprise a nickel-titanium alloy that is configured to self-deploy to the S-shape. In another embodiment, the proximal end and the distal end of the wire may curve laterally toward each other to form a C-shape.

The tacking device may be delivered to a target site using an insertion tool comprising a hollow lumen having an inner diameter configured to receive the wire. The wire is configured to be held in the delivery state when disposed within the hollow lumen. In the delivery state, the wire may be oriented in a substantially longitudinal direction with respect to the insertion tool. The insertion tool maintains the wire in the delivery state.

In use, the graft member may be positioned over a selected region of tissue. The insertion tool may be advanced distally to penetrate through the graft member and through a portion of the tissue. The insertion tool then may be proximally retracted with respect to the tacking device to cause the distal end of the wire to deploy and engage the tissue. Further translation of the insertion tool with respect to the tacking device may cause the proximal end of the wire to deploy and engage the graft member. A stylet loaded into the hollow lumen may abut the proximal end to facilitate retraction of the insertion tool with respect to the tacking device. If desired, multiple tacking devices may be sequentially loaded within the hollow lumen of the insertion tool and then sequentially deployed to secure the tissue to the graft material at multiple different locations. Related procedures may be used without the graft material in order to close a perforation using the tacking device, where both ends of the tacking device engage the tissue.

According to the more detailed aspects, the tacking device preferably comprises one single wire, so it may impose less friction on the interior wall of the insertion tool. It may be easier to load into the insertion tool as well, thereby making loading multiple tacking devices into the insertion tool less time-consuming. Further, the reduction in friction resulting from using a single wire may make it easier to deploy the tacking device from the insertion tool.

Embodiments of the tacking device are also designed in a way to accommodate different thicknesses of tissue. Once fully deployed, if the tissue is thin, in one embodiment the tacking device will simply double back on itself. If the tissue is thick, then the tacking device will stretch out more longitudinally resulting in more of an elongated S shape. In another embodiment, the tacking device will form a closed C shape when the tissue is thin, or may stretch out to form more of an elongated C shape when the tissue is thick.

Optionally, at least one loop member configured to receive a suture may be used for further securing the graft member to the tissue. The loop member may be integrally formed within the wire by bending a portion of the wire 360 degrees so that a loop is formed within the wire, or the loop may be formed by adding an arch-shaped segment of wire to the tacking device. In use, multiple tacking devices comprising loop members may be deployed, and a suture may be threaded through the loop members and actuated in a purse-string fashion.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a plan view of a tacking device.

FIG. 2 is another plan view of a tacking device.

FIG. 3 is another plan view of a tacking device.

FIG. 4 is another plan view of a tacking device.

FIG. 5 is a perspective view of a distal region of an insertion tool and the tacking device of FIG. 1.

FIG. 6 is a perspective, cut-away view illustrating multiple tacking devices in a delivery state.

FIG. 7 is a schematic view illustrating a ventral hernia.

FIG. 8 is a schematic view illustrating a graft member used to cover the ventral hernia of FIG. 7.

FIG. 9 is a schematic view of a method step for treating the ventral hernia of FIG. 5.

FIG. 10 is a side-sectional view taken along line A-A of FIG. 9.

FIG. 11 is a side-sectional view showing multiple tacking devices positioned in deployed states.

FIG. 12 is a schematic view illustrating multiple deployed tacking devices used to treat the ventral hernia of FIG. 7.

FIG. 13 is a side-sectional view illustrating a tacking device in the deployed state engaged with tissue of a certain thickness.

FIG. 14 is a side-sectional view illustrating a tacking device in the deployed stated engaged with tissue of a certain thickness.

FIG. 15 is a plan view of an alternative tacking device.

FIG. 16 is a plan view of another alternative tacking device.

FIG. 17 is a side-sectional view illustrating one method of use of multiple tacking devices of FIG. 16.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patient's anatomy during a medical procedure.

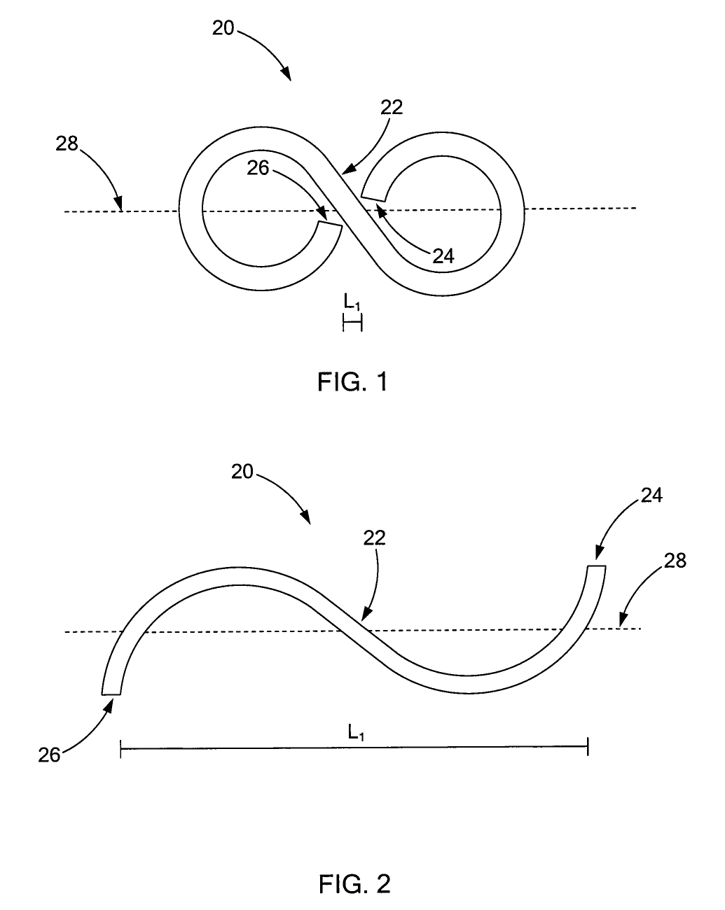

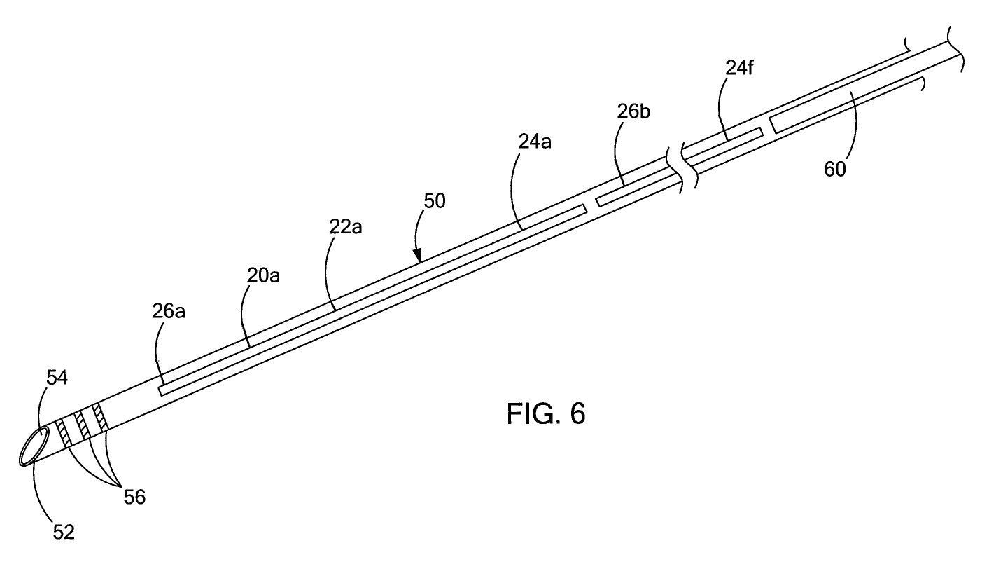

Referring now to FIG. 1, one embodiment of a tacking device 20 is shown constructed in accordance with the teaching of the present invention. In this embodiment, the tacking device 20 comprises a single wire having a proximal end 24 and a distal end 26 connected by an intermediate section 22. In the embodiment of FIG. 1, the proximal end 24 and the distal end 26 are in the deployed state. Since the device is symmetrical, it may be loaded into an insertion tool with either end first (i.e., the proximal and distal ends 24 and 26 are interchangeable), as explained further below. The embodiment of FIG. 1 is operable between a deployed state and a delivery state. As shown in FIG. 1, the natural unbiased state of the wire is the deployed state. In the delivery state, the tacking device 20 is generally straight, thereby aligning itself with a longitudinal axis 28, as shown by tacking device 20a in FIG. 6.

In the embodiment of FIG. 1, the curvature of the proximal and distal ends 24 and 26 spans about 330 degrees (plus or minus 15 degrees) in the deployed state, such that the S shape approximates a figure-eight. In this embodiment, the intermediate section 22 remains generally straight in the deployed state. The proximal and distal ends 24 and 26 of this embodiment extend laterally in different directions from the longitudinal axis 28. Looking at FIG. 1, the curvature of the distal end 26 faces in one direction away from the longitudinal axis 28, while the curvature of the proximal end 24 faces in the opposite direction. The proximal and distal ends 24 and 26 need not be co-planar (e.g., they could be rotated 90 degrees from each other).

The dimensions of the tacking device 20 may be tailored based on a particular surgical procedure, a particular patient's anatomy and/or other factors. However, for illustrative purposes, the longitudinal length of the tacking device 20 preferably ranges from about 0.30 mm to about 0.50 mm in the delivery state, and is most preferably about 0.37 mm. The longitudinal distance L₁ between the ends 24 and 26 may range from about 0 mm to about 0.60 mm, depending on tissue thickness. The diameter of the wire preferably ranges from about 0.008 mm to about 0.024 mm, and most preferably is about 0.016 mm. Such dimensions are provided for reference purposes only and are not intended to be limiting

The tacking device 20 may comprise any suitable shape and material. Solely by way of example, the tacking device 20 may comprise stainless steel or a biocompatible plastic. The tacking device 20 may comprise any shape-memory material, such as a nickel-titanium alloy (nitinol). If a shape-memory material such as nitinol is employed, the tacking device 20 may be manufactured such that it can assume the preconfigured deployed state shown in FIG. 1 upon application of a certain cold or hot medium. More specifically, a shape-memory material may undergo a substantially reversible phase transformation that allows it to “remember” and return to a previous shape or configuration. For example, in the case of nitinol, a transformation between an austenitic phase and a martensitic phase may occur by cooling and/or heating (shape memory effect) or by isothermally applying and/or removing stress (superelastic effect). Austenite is characteristically the stronger phase and martensite is the more easily deformable phase.

In an example of the shape-memory effect, a nickel-titanium alloy having an initial configuration in the austenitic phase may be cooled below a transformation temperature (M_{f}) to the martensitic phase and then deformed to a second configuration. Upon heating to another transformation temperature (A_{f}), the material may spontaneously return to its initial, predetermined configuration, as shown in FIG. 1. Generally, the memory effect is one-way, which means that the spontaneous change from one configuration to another occurs only upon heating. However, it is possible to obtain a two-way shape memory effect, in which a shape memory material spontaneously changes shape upon cooling as well as upon heating.

Alternatively, the tacking device 20 may be made from other metals and alloys that are biased, such that they may be restrained by the insertion tool 50 prior to deployment, but are inclined to return to their relaxed, deployed state upon deployment. Solely by way of example, the tacking device 20 may comprise other materials such as stainless steel, cobalt-chrome alloys, amorphous metals, tantalum, platinum, gold and titanium. The tacking device 20 also may be made from non-metallic materials, such as thermoplastics and other polymers.

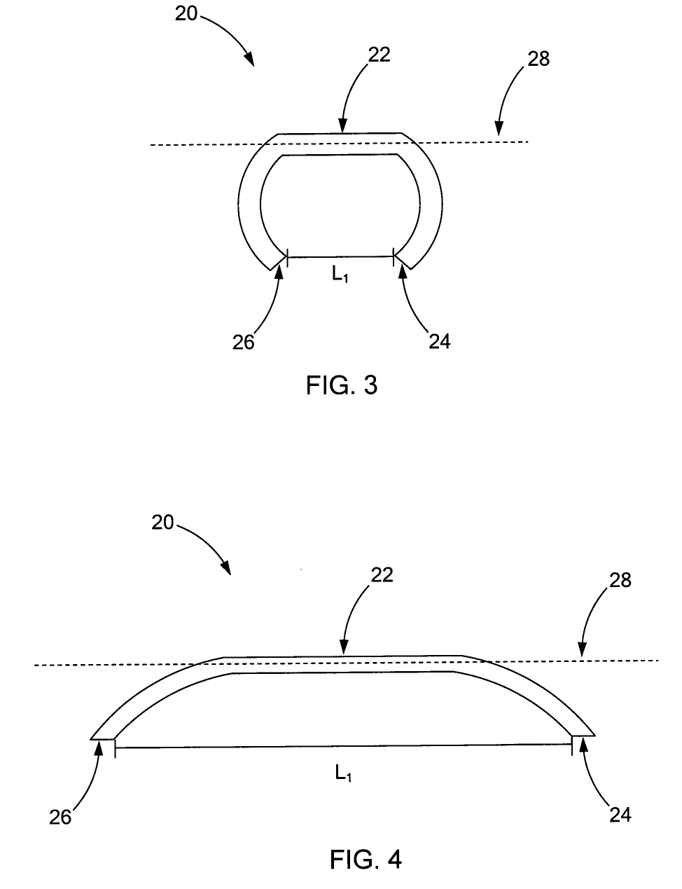

While one embodiment of the tacking device 20 is shown in FIG. 1, the tacking device may comprise various shapes suitable for engaging, penetrating and/or abutting tissue, for purposes explained further below, and need not necessarily assume the deployed shape depicted in FIG. 1. In FIG. 2, a second embodiment of a tacking device 20 is shown constructed in accordance with the teaching of the present invention. In this embodiment, the degree of curvature of the proximal and distal ends 24 and 26 is less than the embodiment of FIG. 1 so that the tacking device 20 forms more of an elongated S shape with the proximal and distal ends 24 and 26 in their deployed states. In FIGS. 3 and 4, a third and fourth embodiment of a tacking device 20 are shown constructed in accordance with the teaching of the present invention. In these embodiments, the proximal and distal ends 24 and 26 move laterally away from the longitudinal axis 28 in the same direction to form a “C” shape. In addition, while the embodiments of FIGS. 1-4 show a tacking device 20 comprised of a single wire, the terms wire or single wire are intended to include monofilament or multifilament wires, the latter of which may be wound, braided, woven, wrapped, or otherwise joined to form a wire. The wire of the tacking device 20 may thus include platinum, gold, nitinol, steel or other radiopaque metal wires wrapped around a core wire, which together form the “single wire” tacking devices as described herein. See, e.g., U.S. Pat. No. 5,330,482, the entire disclosure of which is incorporated herein by reference. The wire of the tacking device 20 could also be two or more wires interwoven together, of the same or different material, for example one or two nitinol wires and one stainless steel wire interwoven together to form the S shape or C shape tacking devices as described above.

Referring to FIGS. 1-6, the proximal and distal ends 24 and 26 each comprise a delivery state, as shown in FIG. 6 below, and further comprise a deployed state, as shown in FIGS. 1-4. In all of the embodiments, each of the ends 24 and 26 comprise a hook-shape in the deployed state. The ends 24 and 26 retroflex preferably between about 90 degrees to about 360 degrees in the deployed state and preferably are circular or semi-circular. In the embodiments depicted in FIGS. 1 and 2, FIG. 1 depicts a curvature of about 330 degrees, while FIG. 2 depicts a curvature of about 120 degrees. Where the ends 24 and 26 have a curvature of about 180 degrees so that the tacking device 20 forms even more of an elongated S shape than depicted in FIG. 2, the proximal and distal ends 24 and 26 are oriented substantially parallel to the intermediate section 22. When the ends 24 and 26 have a curvature of about 330 degrees or greater, the proximal and distal ends 24 and 26 are oriented substantially perpendicular to the intermediate section 22 as shown in FIG. 1. In the embodiments depicted in FIGS. 3 and 4, FIG. 3 illustrates a tacking device with more of a closed C shape, while FIG. 4 shows a tacking device with more of an elongated C shape. The degree of curvature for all embodiments may range anywhere from about 90 degrees to about 360 degrees.

The degree of curvature may also vary based on tissue thickness. For example, FIGS. 13 and 14 depict a tacking device 20 engaging tissues of varying thicknesses t₁. In FIG. 13, where the tissue thickness t₁ is thin, the tacking device 20 forms more of a figure-eight shape as depicted in FIG. 1. In FIG. 14, where the tissue thickness t₁ is thicker, the tacking device 20 forms more of an S shape as depicted in FIG. 2. The intermediate section 22 has been shown generally straight in the embodiments depicted in FIGS. 1-4, although it could be curved.

Further, a longitudinal distance L₁ between the ends 24 and 26 of the tacking device 20 may be varied to engage tissue in a desirable manner. For example, the longitudinal distance L₁ may be dimensioned to be substantially equal to or less than the combined thickness tᵢ and t₂ of a tissue 74 and a graft member 80, respectively, as shown in FIG. 8 below, when the tacking device 20 is retroflexed, thereby providing a desired compressive force upon the tissue 74 and the graft member 80. The overall length of the tacking device 20 also may be varied to engage tissue in a desirable manner.

As noted above, the tacking device 20 may comprise any shape suitable for engaging, penetrating and/or abutting tissue, for purposes explained further below, and need not necessarily assume the curved S shape or curved C shape depicted in FIGS. 1-4. For example, for the embodiments depicted in FIGS. 1-4, the proximal ends 24 and distal ends 26 may move laterally away from the longitudinal axis 28 such that the proximal ends 24 form up to about 90 degree angles from the distal ends 26.

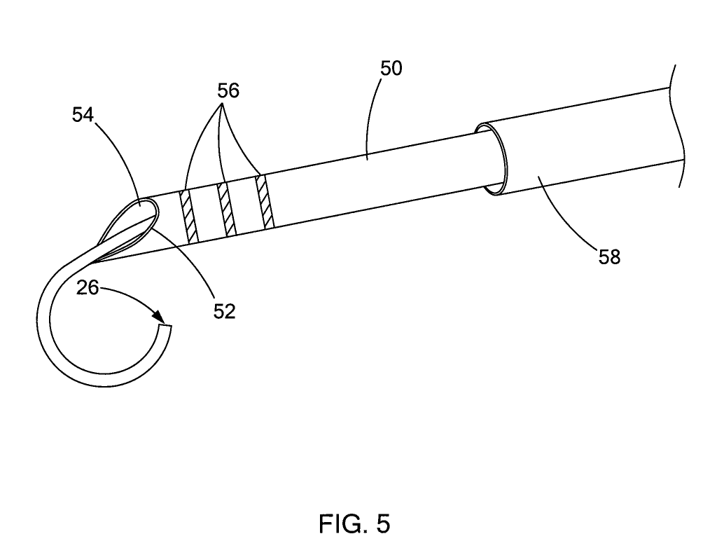

Referring to FIGS. 5-6, one or more tacking devices 20 may be delivered to a target site in a patient's anatomy using an insertion tool 50. In one embodiment, the insertion tool 50 is capable of carrying multiple different tacking devices, such as six tacking devices 20a-20f, as shown in FIG. 12 and described below. In FIG. 6, one complete tacking device 20a is shown in the straightened delivery state, while portions of the distal end 26b of another tacking device 20b, and the proximal end 24f of another tacking device 20f, are also shown. Any embodiment of the tacking device of this invention is generally straight in the delivery state, as depicted in FIG. 6.

In one embodiment, the insertion tool 50 comprises a needle-like body having a sharpened distal tip 52 and a hollow lumen 54, as shown in FIGS. 5-6. The insertion tool 50 may be manufactured from stainless steel or any other suitable material, and may comprise an endoscopic ultrasound (EUS), or echogenic, needle. Solely by way of example, the insertion tool 50 may comprise the EchoTip® Ultrasound Needle, or the EchoTip® Ultra Endoscopic Ultrasound Needle, both manufactured by Cook Endoscopy of Winston-Salem, N.C.

The hollow lumen 54 of the insertion tool 50 may comprise an inner diameter that is larger than an outer diameter of the tacking device 20. The hollow lumen 54 may further comprise an inner diameter that is less than twice the outer diameter of the tacking device 20. One or more tacking devices, such as six tacking devices 20a-20f, may be loaded into the hollow lumen 54 in a delivery state, as shown in FIG. 6. In the delivery state, the proximal and distal ends 24 and 26 of each tacking device 20a-20f may comprise a substantially longitudinally-oriented profile, i.e., oriented along a longitudinal axis of the insertion tool 50.

The multiple tacking devices 20a-20f may be inserted into the hollow lumen 54 of the insertion tool 50 in a sequential manner, whereby the proximal end 24a of the first tacking device 20a may abut the distal end 26b of the second tacking device 20b, as depicted in FIG. 6. The insertion tool 50 maintains the tacking devices in the delivery state. The distal end 26a of the first tacking device 20a may be loaded a distance away from the sharpened distal tip 52 of the insertion tool 50 to prevent inadvertent deployment.

A stylet 60 may be disposed for longitudinal movement within the hollow lumen 54 of the insertion tool 50, as shown in FIG. 6. The stylet 60 may comprise stainless steel or any other suitable material. The stylet 60 is disposed proximal to the proximal end 24f of the final sequential tacking device 20f, as shown in FIG. 6. During use, the insertion tool 50 may be proximally retracted, while the stylet 60 may be held longitudinally steady, to facilitate sequential deployment of each of the tacking devices 20a-20f, as explained further below.

To facilitate the deployment of multiple tacking devices 20, it may be helpful to monitor the degree of retraction of the insertion tool 50. For example, the stylet 50 may comprise one or more markers (not shown), which may be disposed near the proximal end of the stylet 60 so that a physician may determine how far the insertion tool 50 has been retracted. In another embodiment, the stylet 50 may comprise indentations for tactile feel (not shown), which may be disposed at any point along the length of the stylet 60 so that a physician may determine by feel how far the insertion tool 50 has been retracted. Likewise, the handle assembly may comprise stops (not shown) that cooperate with corresponding features disposed near the proximal end of the stylet 60, so that a physician may determine how far the insertion tool 50 has been retracted. Finally, spacers may be employed between the tacking devices 20 to ensure one device is ejected at one time.

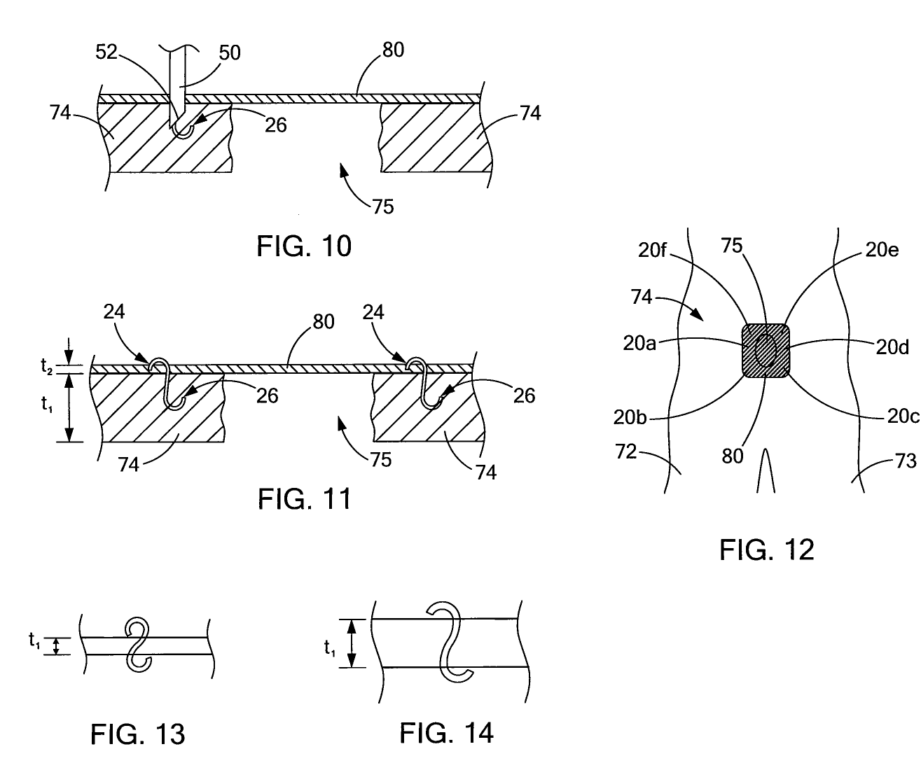

The insertion tool 50 may comprise one or more markers 56, as shown in FIGS. 5-6, which may be disposed near the distal end of the insertion tool 50. The markers 56 may be configured to be visualized under fluoroscopy or other imaging techniques to facilitate location of the distal end of the insertion tool, for example, so that a physician may determine how far the insertion tool 50 has penetrated into tissue 74, as depicted in FIGS. 9-10. Optionally, a sheath member 58 having an inner diameter larger than an outer diameter of the insertion tool 50, as shown in FIG. 5, may be longitudinally advanced over the insertion tool 50, for various purposes explained further below. As will be explained further below, the insertion tool 50 may be used in conjunction with another device, such as an endoscope, and may be delivered through a working lumen of an endoscope or similar device.

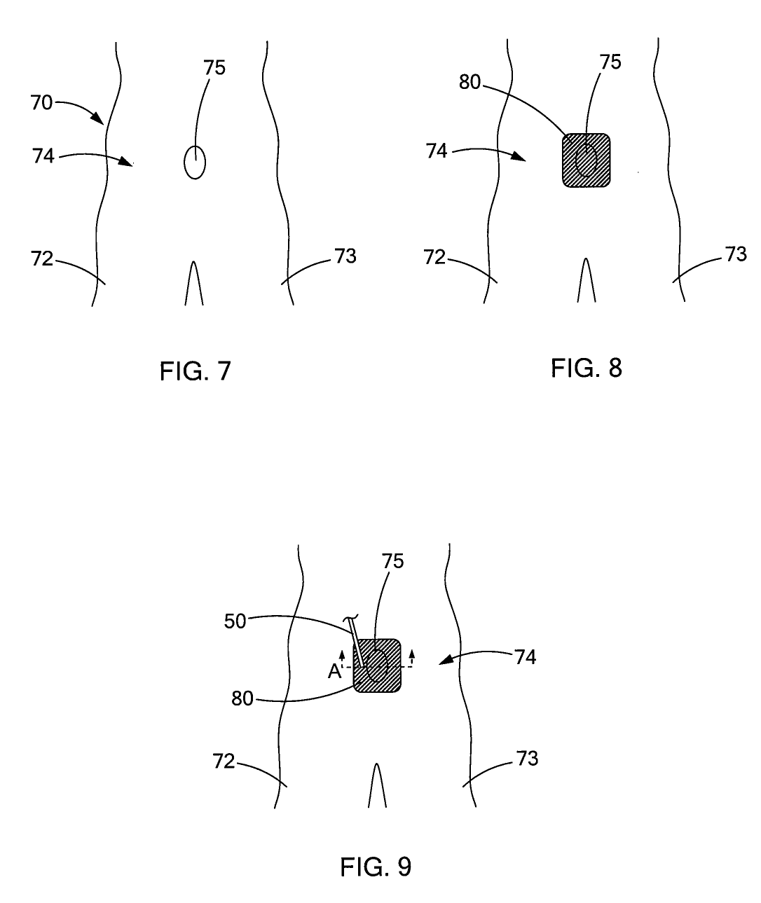

Referring now to FIGS. 7-12, one or more tacking devices 20 described above may be used to facilitate treatment of a perforation 75 using a graft member 80. In the example shown, the perforation 75 is a ventral hernia located in the abdominal wall 74. The right and left legs 72 and 73 of a patient 70 are shown for illustrative purposes. While treatment of a ventral hernia is shown for illustrative purposes, it will be apparent that the tacking devices described herein may be used in a wide range of medical procedures, including but not limited to any exemplary procedures described herein.

The initial stages of the ventral hernia repair may be performed using techniques that are known. Specifically, an open technique or laparoscopic technique may be employed. In an open technique, an incision may be made in the abdominal wall and fat and scar tissue may be removed from the area. A graft member 80 then may be applied so that it overlaps the perforation 75, preferably by several millimeters or centimeters in each direction, as depicted in FIG. 8. In a laparoscopic technique, two or three smaller incisions may be made to access the hernia site. A laparoscope may be inserted into one incision, and surgical instruments may be inserted into the other incision(s) to remove tissue and place the graft member 80 in the same position as the open procedure.

The graft member 80 may comprise any suitable material for covering the perforation 75 and substantially or entirely inhibiting the protrusion of abdominal matter. In one embodiment, the graft member 80 may comprise small intestinal submucosa (SIS), such as SURGISIS® BIODESIGN™ Soft Tissue Graft, available from Cook Biotech, Inc., West Lafayette, Ind., which provides smart tissue remodeling through its three-dimensional extracellular matrix (ECM) that is colonized by host tissue cells and blood vessels, and provides a scaffold for connective and epithelial tissue growth and differentiation along with the ECM components. Preferably, the graft member 80 would be a one to four layer lyophilized soft tissue graft made from any number of tissue engineered products. Reconstituted or naturally-derived collagenous materials can be used, and such materials that are at least bioresorbable will provide an advantage, with materials that are bioremodelable and promote cellular invasion and ingrowth providing particular advantage. Suitable bioremodelable materials can be provided by collagenous ECMs possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa. The graft member 80 may also comprise a composite of a biomaterial and a biodegradable polymer. Additional details may be found in U.S. Pat. No. 6,206,931 to Cook et al., the disclosure of which is incorporated herein by reference in its entirety.

Referring now to FIGS. 9-10, after the graft member 80 has been placed to cover the perforation 75, the insertion tool 50 may be advanced in a distal direction to pierce through the graft member 80, and further may pierce at least partially into the tissue 74 at a first location around the perimeter of the perforation 75. In this example, the insertion tool 50 is carrying six sequential tacking devices 20a-20f, which may be disposed within the hollow lumen 54 of the insertion tool 50 as shown and explained with respect to FIG. 6 above. With each of the tacking devices 20a-20f in the delivery states, the sharpened tip 52 of the insertion tool 50 may be advanced to a predetermined depth into the tissue 74. The markers 56 of FIGS. 5-6 may facilitate in determining how far the insertion tool 50 has penetrated into tissue 74, as depicted in FIG. 10.

In a next step, the stylet 60 of FIG. 6 may be held steady with respect to the insertion tool 50, while the insertion tool 50 is retracted in a proximal direction. This causes the distal end 26 of the most distal tacking device 20a to extend distally to the sharpened tip 52 of the insertion tool 50, as depicted in FIG. 10. When the distal end 26 is no longer radially constrained by the insertion tool 50, it may assume its predetermined deployed state in which it may engage, penetrate and/or abut the tissue 74. As the insertion tool 50 further is retracted proximally with respect to the tacking device 20a, the proximal end 24 may assume its predetermined deployed state when it is no longer radially constrained, as shown in FIG. 11. In the deployed state, the proximal end 24 may engage, penetrate and/or abut the graft member 80 and optionally penetrate into the tissue 74. In this manner, the tacking device 20a helps secure the graft material 80 against the tissue 74. In particular, the substantially 360-degree hook-shaped configuration of the proximal end 24 may urge the graft member 80 in a distal direction towards the tissue 74.

After the first tacking device 20a has been deployed, the insertion tool 50 may be repositioned to deploy another tacking device around the perimeter of the perforation 75. Each subsequent tacking device 20b-20f may be deployed in the same manner as the tacking device 20a. In this manner, the tacking devices 20a-20f may secure the graft member 80 around the perimeter of the perforation 75, as shown in FIG. 12. As will be apparent, greater or fewer tacking devices may be used, and the positioning of the tacking devices may be varied to optimize securing the graft member 80 to the tissue 74 in order to substantially seal the perforation 75.

Optionally, the sheath member 58 of FIG. 5 may be longitudinally advanced over the insertion tool 50, for example, if needed to protect the sharpened distal tip 52 of the insertion tool 50 while the insertion tool 50 is being repositioned. Further, the sheath member 58 may be advanced distally over the insertion tool 50 to facilitate deployment of the proximal end 24. For example, the sheath member 58 may periodically push against the graft member 80, thereby temporarily urging the graft member 80 and/or the tissue 74 in a distal direction. At this time, the sheath member 58 may be held steady while the insertion tool 50 is retracted proximally to deploy the proximal end 24 at a location proximal to the compressed tissue 74 and graft member 80. Once the proximal end 24 has been deployed, the compressive force applied by the sheath member 58 may be removed so that the deployed proximal end 24 may engage the graft member 80 and the tissue 74.

In the embodiment of FIGS. 7-12, the tissue 74 illustratively comprises a thickness t₁, while the graft member 80 comprises a thickness t₂. The distal end 26 may be deployed entirely within the tissue 74, as depicted in FIG. 11, or alternatively may be deployed substantially distal to the tissue 74 while abutting or piercing through a distal edge of the tissue 74. In the latter embodiment, the longitudinal distance L₁ between the ends 24 and 26 of the tacking device 20 may be dimensioned to be substantially equal to, or slightly less than, the combined thickness t₁+t₂ of the tissue 74 and the graft member 80. The longitudinal distance L₁ may be otherwise sized and configured, as desired, to apply desired forces upon the graft member 80 and the tissue 74.

While FIGS. 7-12 have illustrated the use of one or more tacking devices 20 for covering a perforation 75 formed in the ventral abdominal wall, the tacking devices disclosed herein may be useful in many other procedures. Solely by way of example, one or more tacking devices 20 may be used to treat perforations in a visceral wall, such as the stomach wall. In such cases, a suitable insertion device, such as an endoscope, may be advanced through a bodily lumen such as the alimentary canal to a position proximate the target location. One or more components may be advanced through a working lumen of the endoscope. To close the perforation, the graft member 80 may cover the perforation and may be secured in a position overlapping the perforation using the one or more of the tacking devices 20, which may be deployed using the techniques described hereinabove.

As mentioned above, the tacking devices of this invention may also be used for closing an opening in tissue. Referring to FIGS. 7-14, the same steps may be utilized to secure a graft material to tissue may be used to have the tacking device directly engage a perforation in tissue instead of engaging a perforation and a graft material. When the tissue is thin, as depicted in FIG. 13, then one embodiment of the tacking device will form more of a figure-eight shape in the deployed state as depicted in FIG. 1. When the tissue is thick, as depicted in FIG. 14, one embodiment of the tacking device will form more of an elongated S shape as shown in FIG. 2.

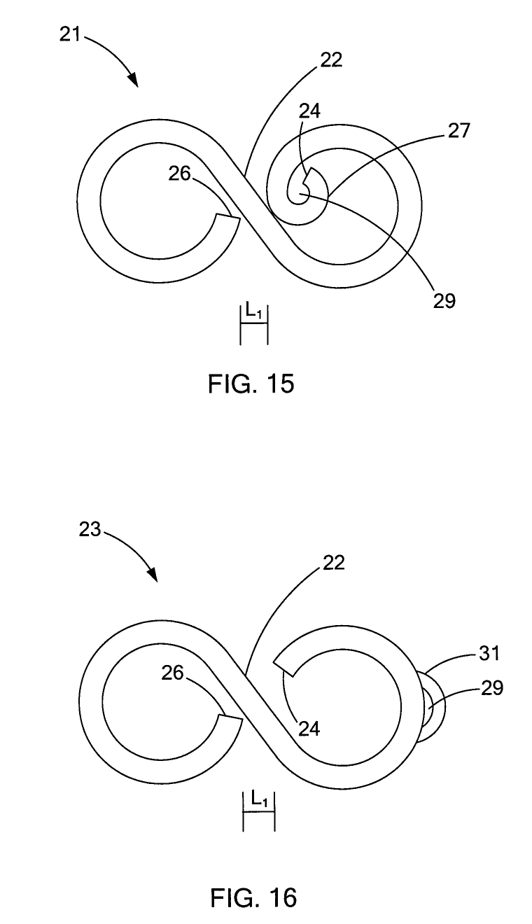

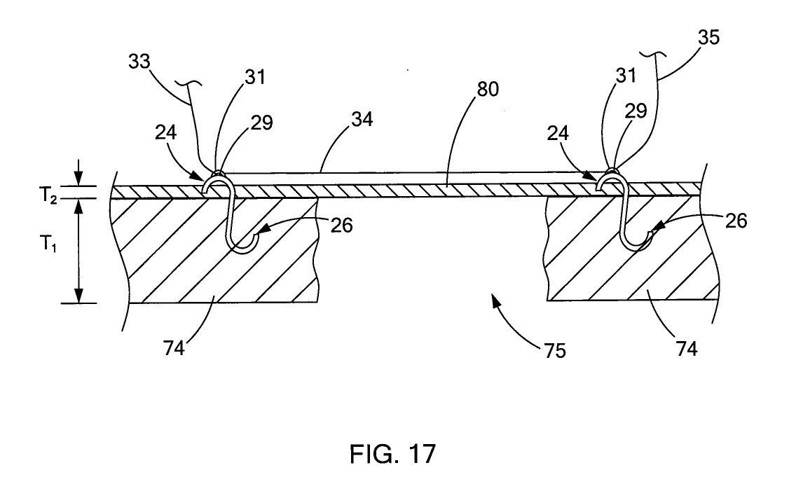

Referring now to FIGS. 15 and 16, in an alternative embodiment, tacking devices 21 or 23 may comprise one or more features for facilitating suturing, and preferably purse-string suturing. The tacking devices 21 and 23 are similar to the tacking device 20 of FIGS. 1-4, except as noted below. The tacking devices 21 and 23 comprise proximal and distal ends 24 and 26, respectively. In one embodiment, the tacking device 21 comprises a proximal end and a distal end with a loop 27 formed at any point along its length, preferably formed as shown in FIG. 15 by bending a region of the wire that is disposed near the proximal end. The tacking device may be bent to form an annular loop member 27 having an aperture 29. In the embodiment of FIG. 16, a segment of wire may be added to tacking device 23 to form an annular loop member 31 having an aperture 29. A suture may be threaded through the aperture of the loop member 31, for example, as shown in FIG. 17 below. While the loop member 31 of FIG. 16 is shown substantially near the proximal end 24, it may be placed closer to the distal end 26 of the tacking device 23.

Referring now to FIG. 17, an exemplary method of using the tacking device 23 is shown. In one step, a graft member 80 may be placed over a perforation 75, and multiple tacking devices may be deployed using an insertion device to secure the graft member 80 to the tissue 74, as explained in detail above with respect to FIGS. 7-12. In the embodiment of FIG. 17, multiple tacking devices may be linked together by a single suture 34, which may be slidably coupled through the loop members of each of the tacking devices 23, as generally shown in FIG. 17. There are two free ends 33 and 35 of the suture 34, which may be independently tensioned to facilitate closure of the perforation 75.

Preferably, multiple tacking devices having loop members are sequentially positioned around the perforation 75 in a semi-annular or annular shape, for example, as shown above in FIG. 12. The ends of the suture are then tensioned to reduce the distance between the tacking devices and compress the tissue 74 around the perforation 75. The suture ends may be secured to maintain the compression of the tissue 74 using any suitable technique such as by forming a knot or using clamps, rivets and the like.

Further, in lieu of the loop members described herein, other mechanisms for engaging and/or retaining sutures may be integrally formed with the tacking device or externally attached thereto. Solely by way of example, such suture retaining mechanisms are explained in pending U.S. patent application Ser. No. 11/946,565, filed Nov. 28, 2007, and U.S. patent application Ser. No. 12/125,528, filed May 22, 2008, the entire disclosures of which are hereby incorporated by reference in their entirety.

Various types of sutures may be used in conjunction with embodiment of FIGS. 15-17. For example, synthetic sutures may be made from polypropylene, nylon, polyamide, polyethylene, and polyesters such as polyethylene terephthalate. These materials may be used as monofilament suture strands, or as multifilament strands in a braided, twisted or other multifilament construction.

While the examples shown above have illustratively described a tacking device that may be useful for coupling a graft member to tissue to cover and seal a perforation, the tacking devices 20, 21 and 23 may be used in other procedures. As noted above, the tacking devices 20, 21 and 23 may be used to treat bodily walls during translumenal procedures. Further, the tacking devices 20, 21 and 23 may be used to secure a graft member to tissue for reconstructing local tissue, and the like.

In yet further applications within the scope of the present embodiments, the tacking devices 20, 21 and 23 need not be used for coupling a graft member to tissue. For example, the tacking devices 20, 21 and 23 may be used in an anastomosis procedure. In order to create an anastomosis, for example, multiple tacking devices 20, 21 or 23 may be deployed in a circular manner to couple a proximal vessel, duct or organ to a distal vessel, duct or organ. In such cases, a suitable insertion device, such as an endoscope, may be advanced through a bodily lumen such as the alimentary canal to a position proximate the target location. One or more components, such as the insertion tool 50, may be advanced through a working lumen of the endoscope. The distal end of the insertion tool 50 may be viewed under fluoroscopy, or via optical elements of the endoscope, or by some other visualization technique. Under suitable visualization, multiple tacking devices then may be delivered at one time, for example, using the insertion tool 50. Then, a hole may be punched through the middle of the deployed tacking devices to create a flow path between the proximal and distal vessels/ducts/organs. It will be apparent that still further applications of the tacking devices 20, 21 and 23 are possible. Moreover, the insertion tool 50 may be used with or without an endoscope or similar device.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. A medical apparatus for coupling a graft member to bodily tissue, the medical apparatus comprising:
a single wire having a proximal end and a distal end;
the wire being flexible and flexing between delivery and deployed states, the proximal end and the distal end each having a curved shape in the deployed state, the curvatures of the proximal and distal ends being oriented relative to one another to form an S-shape in the deployed state; and
the distal end of the wire being configured to engage tissue at a first location in the deployed state, and the proximal end of the wire being configured to engage the graft member in the deployed state to secure the graft member to the tissue.

2. The apparatus of claim 1, wherein the curvatures of the proximal and distal ends are adjustable to adjust the distance between the locations where the first and second ends engage the tissue.

3. The apparatus of claim 1, wherein the wire is formed of a resilient material, and wherein the natural unbiased state of the wire is the deployed state.

4. The apparatus of claim 1, wherein the wire has a length ranging from about 0.30 mm to about 0.50 mm.

5. The apparatus of claim 1, wherein the curvatures of the proximal and distal ends range over an arc of about 90 degrees to about 360 degrees.

6. The apparatus of claim 1 further comprising at least one loop member having an aperture configured to receive a suture for further securing the graft member to the tissue.

7. The apparatus of claim 6, wherein the loop member is integrally formed with the wire by deforming a portion of the wire about 360 degrees.

8. A medical apparatus for coupling a graft member to bodily tissue, the medical apparatus comprising:
a wire having a proximal end and a distal end, the wire operable between delivery and deployed states;
the wire being generally straight in the delivery state to define a longitudinal axis;
the proximal and distal ends of the wire moved laterally away from the longitudinal axis in the deployed state; and
the proximal and distal ends of the wire extending in different lateral directions away from the longitudinal axis in the deployed state.

9. The apparatus of claim 8 further comprising an intermediate section disposed between the distal and proximal ends, the intermediate section being straight.

10. The apparatus of claim 8, wherein the proximal and distal ends are curved in the deployed state, and wherein the curvatures of the proximal and distal ends range over an arc of about 90 degrees to about 360 degrees.

11. The apparatus of claim 8, wherein the wire is formed of a resilient material, and wherein the natural unbiased state of the wire is the deployed state.

12. The apparatus of claim 8, wherein the curvatures of the proximal and distal ends are oriented relative to one another to form an S-shape in the deployed state.

13. The apparatus of claim 8, wherein the wire has a length ranging from about 0.30 mm to about 0.50 mm.

14. A system suitable for coupling a graft member to bodily tissue, formed by a plurality of the medical apparatuses as recited in claim 8, the system further comprising:
an insertion tool comprising an insertion lumen having an inner diameter configured to receive the plurality of wires such that the proximal and distal ends of adjacent wires abut each other within the insertion lumen; and
a stylet slidably disposed within the insertion lumen and abutting a wire of the plurality of wires and operable from a proximal end of the stylet to deliver a wire beyond the distal end of the insertion tool.

15. The system of claim 14 wherein the hollow lumen has an inner diameter that is less than twice the diameter of the wires.

16. The system of claim 14 wherein the graft member is used to cover a perforation, wherein one or more wires are deployed at one or more locations around a perimeter of the perforation to secure the graft member to tissue surrounding the perforation.

17. The system of claim 14 wherein the proximal ends and the distal ends self-deploy to form an S-shape when no longer constrained by the hollow lumen of the insertion tool.

18. The system of claim 14, wherein the insertion tool is a delivery needle having a distal end adapted to pierce tissue.

19. The system of claim 14 further comprising a sheath member adapted to be advanced over the insertion tool and configured to abut the tissue or the graft member.
